(19) United States
(12) Patent Application Publication
(10) Pub. No.: US 2006/0008485 A1
(43) Pub. Date: Jan. 12, 2006

(54) Packaged cosmetic compositions and related methods

(76) Inventors: James Joseph Ferone, Bridgewater, NJ (US); Gale McElroy Reinhart, Middletown, NJ (US); Neil D. Scancarella, Wyckoff, NJ (US); Michael David Helman, Edison, NJ (US)

Correspondence Address: Julie Blackburn;Revlon Consumer Products Corporation, 237 Park Avenue, New York, NY, 10017, US

(21) Appl. No.: 10/889,522

(22) Filed: Jul. 12, 2004

(51) Int. Cl. A61K 8/02

(52) U.S. Cl. 424/401

(57) ABSTRACT

A packaged powder-to-liquid-to-powder composition, a method for providing transfer resistant or long wearing cosmetic compositions, and a method for improving tone or laxity of skin.

### TECHNICAL FIELD

The invention is in the field of packaged cosmetic compositions and their use in treating keratinous surfaces to color, condition, beautify, or otherwise cosmetically improve the appearance of such surfaces.

### BACKGROUND OF THE INVENTION

Cosmetics companies are always searching for new vehicles and components for delivering beneficial actives to keratinous surfaces such as skin, coloring skin to minimize the appearance of skin imperfections, or otherwise treating skin for improvement. For example, face powders are very popular with many women. They are typically used after foundation makeup, to blot the excess makeup and to absorb oil and sebum excreted by the skin. Some women use powders alone as a foundation makeup, but as the powder consists mostly of powder and pigment particles, these particles are easily removed from skin and may cake or look unnatural on the skin. Many women like the oil and sebum blotting benefit of powder, but object to applying powder on top of foundation makeup because they feel that the face may look too matte or overly made up.

There is much interest in providing a powder makeup that will provide the coverage of foundation in minimizing the appearance of skin imperfections, give a more even tone to the skin, and at the same time providing the benefits of powders in absorbing oil and sebum and providing a sheer, finished look to the made up face.

It has been discovered that formulating a type of cosmetic composition and packaging it in a composition-compatible delivery system that interacts with the formula will provide a foundation makeup, skin treatment agent, or other composition that will deliver ingredients to the skin in a powder-to-liquid-to-powder form that provides the beneficial effects of both powders and liquid makeups, but without many of the drawbacks.

It is an object of the invention to provide a packaged cosmetic composition comprised of a powder-to-liquid-to-powder cosmetic composition in a package comprised of a container for containing the composition and an applicator for applying the composition which causes the powder to become liquid upon application, when then dries to a powder after application onto the skin.

It is a further object to provide a method for improving the laxity or tone of facial skin comprising applying to such skin a powder-to-liquid-to-powder composition.

It is a further object of the invention to provide a method for preparing transfer resistant or long wearing cosmetic compositions.

### SUMMARY OF THE INVENTION

The invention comprises a packaged powder-to-liquid-to-powder cosmetic composition wherein the composition in the package is in the powder form and comprises a powder-to-liquid effective amount of at least one porous particulate having contained within at least one liquid solvent expressible from the particulate upon application of pressure; and the package comprises a container, and an applicator for the composition that is operable to pick up the composition in its powder form and upon application of the powder to the keratinous surface, exert pressure sufficient to express the liquid from the porous particulate causing the powder to form a liquid upon application to the keratinous surface, which dries to a powder finish.

The invention further comprises a method for improving laxity and/or tone of skin comprising applying to said skin a composition one porous particulate having contained within at least one liquid solvent expressible from the particulate upon application of pressure, and upon application, providing pressure sufficient to express the liquid solvent from the porous particulate to form a liquid film on the skin that, when dry, improves the laxity and/or tone of the skin.

The invention further comprises a method for providing a transfer resistant or long wearing finish on skin by applying a color cosmetic composition containing at least one porous particulate having contained therein at least one liquid solvent expressible from the particulate upon application of pressure, and upon application, providing pressure sufficient to express the liquid solvent from the porous particulate to form a liquid film on the skin that dries to a transfer resistant or long wearing finish.

### BRIEF DESCRIPTION OF THE DRAWINGS

FIG. 1: illustrates a cosmetic compact, one form of package that may be suitable for use with the packaged cosmetic composition of the invention.

FIG. 2: illustrates a jar-type compact, another type of package suitable for containing the packaged cosmetic composition of the invention.

FIG. 2A: shows the jar-type compact of FIG. 2 in side view, showing how the lid is affixed to the base.

FIG. 2B: show the jar-type compact of FIG. 2 with the lid removed from the base.

FIG. 3: illustrates a standard bottle or jar having a cap, another package suitable for containing the packaged cosmetic composition of the invention.

FIG. 4: depicts a number of different types of packages which are in the vial form where the cosmetic is stored in the vials and applied with the applicators shown.

FIG. 4A: illustrates a dual ended package comprising two vials for containing the cosmetic composition and a dual ended cap/rod/applicator assembly having two applicators extended from the cap on opposite ends, for use in applying the cosmetic contained in the two vials.

FIG. 4B: illustrates a vial having a single cap/rod/applicator assembly where the vial contains the cosmetic and the applicator is suitable for applying the cosmetic to the desired keratinous surface.

FIG. 5: illustrates another type of jar for containing the cosmetic, having a lid with pores through which the powder can be shaken when the jar is manipulated, a brush applicator suitable for use in applying the cosmetic, and a closure for the jar.

FIG. 6: illustrates various types of applicators that may be used to apply the cosmetic used in the invention.

FIG. 6A: is a single edge foam or sponge applicator.

FIG. 6B: is a dual ended foam or sponge applicator.

FIG. 6C: is a sponge.

FIG. 7: illustrates a number of suitable brushes for applying the powder cosmetic of the invention.

FIG. 7A: is a cap/rod/brush assembly showing a brush much like a nail enamel brush, suitable for applying the cosmetic used in the invention to small surfaces such as cuticles.

FIG. 7B: depicts a number of different brushes suitable for applying the powder cosmetic to the desired keratinous surface.

### DETAILED DESCRIPTION

I. The Powder-to-Liquid-to-Powder Composition

The first embodiment of the invention is directed to a packaged powder-to-liquid-to-powder cosmetic composition where the composition, when in the package, is in the powdery form, but when applied to the keratinous surface with the applicator becomes a liquid on the skin. The conversion of the powder to a liquid upon application to the keratinous surface with an appropriate applicator is due to the presence of a powder-to-liquid effective amount of porous particulates having solvent contained therein in the composition. While the majority of the solvent remains within, or imbibed, within the porous particulates while the composition is in the resting state, the solvent is capable of being expressed from the particulates upon application of a suitable amount of pressure, such as that applied by an applicator.

The term “powder-to-liquid-to-powder” means that the composition exists as a powder, visually, when observed in the package in its resting state, or when picked up with an applicator prior to application to a keratinous surface. However, upon application to the keratinous surface with an appropriate applicator, enough pressure is exerted to cause the powder to become liquid as it is applied to the keratinous surface. Thereafter, the liquid dries within a reasonable period of time and forms a very sheer dried film on the skin that may appear to some users as a light powdery finish.

The term “powdery form” means that the composition visually appears to be in the loose, free flowing or pressed powder form.

The term “powder-to-liquid” with respect to the amount of porous particulates containing solvent therein, means that the amount of porous particulates containing solvent within must be present in an amount sufficient to cause the composition to exist in a powder form when in the resting state, and a liquid when applied to the keratinous surfaced using an applicator that exerts sufficient pressure to cause the solvent contained within the particulates to exude out to cause the composition to form a liquid upon application.

The term “porous particulate” means a particle that has internal channels or interstices, or lamella, sufficient to imbibe both aqueous or non-aqueous solvents or both, yet be capable of releasing them or causing them to exude from the channels or interstices upon application of a suitable pressure, such as that applied with an appropriate cosmetic applicator.

A. The Porous Particulates

A variety of porous particulates are suitable for use in making the cosmetic composition in the package, or for use in practicing the methods of the invention. Suggested ranges of porous particulates containing solvent are from about 2-98%, preferably about 5-85%, more preferably about 8-80% by weight of the total composition.

Suitable particulates include, but are not limited to, cellulosics such as starches, crosspolymers, resins, silica or silicates, organic or silicone elastomers, and the like. So long as the porous particulate contains internal channels, interstices, or lamella that are capable of imbibing aqueous or non-aqueous solvents that can release the solvents upon application of pressure, the particulates are suitable for use in the composition. The porous particulates may be in the form of platelets that are in lamellar form as well as in the form of particulates containing open cells, interconnected channels, or interstices. The channels in the porous particulate must be, at least partially, open celled such that the solvent present in the particulate can migrate within the channels or interstices, yet be expressed from the channels upon application of suitable pressure. The solvent may be expressed from the particulate by compression in the case where the particulate is compressible, or by capillary action in the case where the porous particulate is not necessarily compressible. In the latter case, the pressure that may be exerted by the suitable applicator is a capillary action.

The porous particulates may be polymeric or non-polymeric in nature, and if polymeric, the polymers may contain polymerized organic monomers, silicone monomers, or combinations thereof. The porous particulates are generally water insoluble and may have particle sizes ranging from about 20 to 1000, preferably about 50-500, more preferably about 100-300 microns. In the most preferred embodiment of the invention, some of the porous particulates that may be used have a porosity ranging from about 50 to 500, preferably from about 90-300, more preferably about 100-250 ml/100 grams as measured by the oil absorbance test using castor oil. At least some of the most preferred particles (in particular the cross polymers) may have a density ranging from about 0.08 to 0.55 g/cm³.

1. Cellulosics

Suitable cellulosics are polymers containing repeating cellulose units, such as starches or modified starches, either as homopolymers or copolymerized with other cellulose monomers or organic monomers. Such cellulosics may also contain alkali metal or alkaline earth metal substituents. The cellulosics may be substituted with one or more groups that confer hydrophobicity or hydrophilicity. Examples of suitable cellulosics include starch, starch substituted with C₁₋₁₀ alkyl or alkoxy groups including methyl, ethyl, propyl, methoxy, ethoxy, propoxy, etc., or starch substituted with alkali or alkaline earth metals such as sodium, potassium, magnesium, aluminum, and so on. Also suitable are cellulosics such as starch that may be copolymerized with succinimates, succinates, or succinimides, or derivatives thereof, including materials such as aluminum starch octenylsuccinate, and the like. Particularly preferred starches are hydroxypropyl starch, hydroxyethyl starch, sodium carboxymethyl starch, aluminum starch octenylsuccinate, corn starch, rice starch, microcrystalline cellulose, maltodextrin, aluminum starch, dextran, glyceryl starch, and the like.

2. Crosspolymers

A wide variety of crosspolymers are also suitable, including organic polymers, silicone polymers, or copolymers of organic and silicone monomers. The term “crosspolymer” generally means a polymer containing groups that have crosslinked. The crosslinking will cause the polymer to form a matrix having inner channels or interstices that are capable of imbibing solvent.

Organic crosspolymers include polymers of polymerized ethylenically unsaturated monomers where at least some of the monomers have crosslinkable groups which crosslink during or soon after polymerization of the polymer. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on.

In some cases, the crosspolymer contains one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 1 to 20, more preferably 1 to 8 carbon atoms. If desired, the aliphatic alcohol may have one or more hydroxy, carboxy, or carboxylic acid groups. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include, but are not limited to, those of the formula: 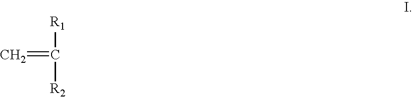
wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM or OCOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl, carboxy, carboxylic acid, or other types of groups, or [(CH₂)ₘO]ₙH wherein m is 1-20, and n is 1-200.

More specific examples include the monofunctional ethylenically unsaturated monomer is of Formula I, above, wherein R₁ is H or a C₁₋₃₀ alkyl, and R₂ is COOM or OCOM wherein M is a C₁₋₃₀ straight or branched chain alkyl which may be substituted with one or more hydroxy groups or other types of crosslinkable groups.

Further examples include where R₁ is H or CH₃, and R₂ is COOM wherein M is a C₁₋₁₀ straight or branched chain alkyl which may be substituted with one or more hydroxy groups.

Di-, tri- and polyfunctional monomers, as well as oligomers, of the above monofunctional monomers may also be used to form the crosspolymer. Suitable difunctional monomers include those having the general formula: 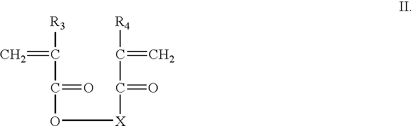
wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference.

Also suitable are polymers formed from the monomer of Formula I, above, which are cyclized, in particular, cycloalkylacrylate polymers or copolymers having the following general formulas: 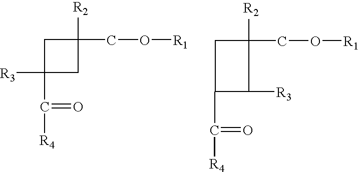
wherein R₁, R₂, R₃, and R₄ are as defined above. Typically such polymers are referred to as cycloalkylacrylate polymers.

The monomers mentioned herein can be polymerized with various types of organic groups such as propylene glycol, isocyanates, amides, etc.

One type of organic group that can be polymerized with the above monomers includes a urethane monomer. Urethanes are generally formed by the reaction of polyhydroxy compounds with diisocyanates, as follows: 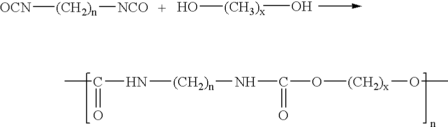
wherein x is 1-1000.

Another type of monomer that may be polymerized with the above comprise amide groups, preferably having the the following formula: 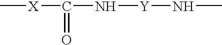
wherein X and Y are each independently linear or branched alkylene having ₁₋₄₀ carbon atoms, which may be substituted with one or more amide, hydrogen, alkyl, aryl, or halogen substituents.

Another type of organic monomer may be alpha or beta pinenes, or terpenes, abietic acid, and the like.

One additional type of synthetic organic polymer that may be used in the compositions of the invention is obtained by polymerizing ethylenically unsaturated monomers which comprise vinyl ester groups either alone or in combination with other monomers including silicon monomers, other ethylenically unsaturated monomers, or organic groups such as amides, urethanes, glycols, and the like. The various types of monomers or moieties may be incorporated into the film forming polymer by way of free radical polymerization, addition polymerization, or by formation of grafts and blocks which are attached to the growing polymer chain according to processes known in the art. Preferably the film forming polymer is an organic synthetic polymer obtained by polymerizing ethylenically unsaturated monomers comprised of vinyl ester groups and optionally organic or silicon groups or other types of ethylenically unsaturated monomers.

Other types of porous particulates may be polymerized and crosslinked polymers having one or more vinyl ester monomers having the following general formula: 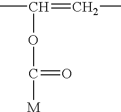
wherein M is H, or a straight or branched chain C₁₋₁₀₀ alkyl, preferably a C₁₋₅₀ alkyl, more preferably a C₁₋₄₅ alkyl which may be saturated or unsaturated, or substituted or unsubstituted, where the substituents include hydroxyl, ethoxy, amide or amine, halogen, alkyloxy, alkyloxycarbonyl, and the like. Preferably, M is H or a straight or branched chain alkyl having from 1 to 30 carbon atoms. The polymeric porous particulate may be a homopolymer or copolymer having the vinyl ester monomers either alone or in combination with other ethylenically unsaturated monomers, organic groups, or silicon monomers.

Suitable other monomers that may be copolymerized with the vinyl ester monomer include those having siloxane groups, including but not limited to those of the formula: 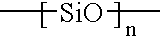
wherein n ranges from 1-1,000,000. The silicon monomers are preferably polymerized into a siloxane polymer then attached to the polymer chain by attaching a terminal organic group having olefinic unsaturation such as ethylene or propylene, to the siloxane, then reacting the unsaturated group with a suitable reactive site on the polymer to graft the siloxane chain to the polymer.

Various types of organic groups may be polymerized with the vinyl ester monomers including but not limited to urethane, amide, polyalkylene glycols, and the like as set forth above.

The vinyl ester monomers may also be copolymerized with other ethylenically unsaturated monomers that are not vinyl esters, including those set forth above.

Most preferred is where the crosspolymer is a polymer of crosslinked methyl methacrylate or crosslinked polystyrene. Particular preferred is a crosslinked polymethylmethacrylate having the INCI name methyl methacrylate crosspolymer, which may be purchased from Presperse Inc., in Piscataway, N.J., and is available under the tradename Ganzpearl.

3. Resins

Also suitable as the porous particulate are various resins including silicone resins, organic resins, or copolymers thereof, so long as the resin exhibits at least some internal channels and is capable of imbibing the solvent. In the context of this invention, the term “resin” will mean a siloxane containing enough cross-linking to provide a porous particulate having internal channels. In some cases such resins may also provide substantive, film forming properties.

Typically silicone resins are at least partially crosslinked and include those referred to as T or Q resins. The term “T” generally means “trifunctional siloxy unit” and in standard silicone nomenclature a “T” unit has the general formula:
R₁SiO_{3/2}
wherein R₁ is C₁₋₃₀, preferably C₁₋₁₀, more preferably, C₁₋₄ straight or branched chain alkyl, which may be substituted with phenyl or one or more hydroxyl groups; phenyl; alkoxy (preferably C₁₋₂₂, more preferably C₁₋₆ alkyl); or hydrogen. The SiO_{3/2} designation means that the silicon atom is bonded to three oxygen atoms when the unit is copolymerized with one or more of the other units. For example when R₁ is methyl the resulting trifunctional unit is of the formula:

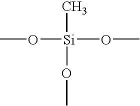

When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:
SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

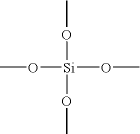

The resin may contain only T or Q units, or may be copolymerized with other siloxane units such as M or D units.

The term “monofunctional unit” or “M” means a siloxy unit that contains one silicon atom bonded to one oxygen atom, with the remaining three substituents on the silicon atom being other than oxygen. In particular, in a monofunctional siloxy unit, the oxygen atom present is shared by 2 silicon atoms when the monofunctional unit is polymerized with one or more of the other units. In silicone nomenclature used by those skilled in the art, a monofunctional siloxy unit is designated by the letter “M”, and means a unit having the general formula:
R₁R₂R₃SiO_{1/2}
wherein R₁, R₂, and R₃ are each independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆ alkyl; or hydrogen. The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula:

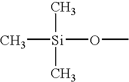

When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D′” designation is sometimes used, which indicates a substituent other than methyl. For purposes of this disclosure, a “D” unit has the general formula:
R₁R₂SiO_{2/2}
wherein R₁ and R₂ are defined as above. The SiO_{2/2} designation means that the silicon atom in the difunctional unit is bonded to two oxygen atoms when the unit is polymerized with one or more of the other units. For example, when R₁, R₂, are methyl the resulting difunctional unit is of the formula: 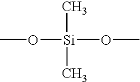
When this difunctional unit is polymerized with one or more of the other units the silicon atom will be bonded to two oxygen atoms, i.e. will share two one-halves of an oxygen atom.

The siloxane resins that form suitable porous particulates generally comprise a majority of T or Q units, either alone or in combination with minor amounts of M or D units, the phrase “major amount” meaning that the T or Q units in the polymer are present such that the resulting polymer has sufficient porosity. The term “minor amount” means that the M or D units, if present, are not present in an amount that provides a particulate that is does not have the required degree of porosity

T or MT silicones are often referred to as silsesquioxanes, and in the case where M units are present methylsilsesquioxanes. One type of T silicone that may be suitable for use as the porous particulate has units of the following general formula:
(R₁SiO_{3/2})x
where x ranges from about 1 to 100,000, preferably about 1-50,000, more preferably about 1-10,000, and wherein R₁ is as defined above. Such MT silicones are generally referred to as polymethylsilsesquioxane which are silsesquioxanes containing methyl groups.

Examples of specific polysilsesquioxanes that may be used are manufactured by Wacker Chemie under the Resin MK designation. This polysilsesquioxane is a polymer comprise of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable polysilsesquioxanes that may be used as the porous particulate include those manufactured by Shin-Etsu Silicones and include the “KR” series, e.g. KR-220L, 242A, and so on. These particular silicone resins may contain endcap units that are hydroxyl or alkoxy groups which may be present due to the manner in which such resins are manufactured.

Also suitable are MQ resins, which are siloxy silicate polymers having the following general formula: 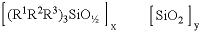
wherein R¹, R² and R³ are each independently a C₁₋₁₀ straight or branched chain alkyl or phenyl, and x and y are such that the ratio of (R¹R²R³)₃SiO_{1/2} units to SiO₂ units ranges from about 0.5 to 1 to 1.5 to 1. Preferably R¹, R² and R³ are a C₁₋₆ alkyl, and more preferably are methyl and x and y are such that the ratio of (CH₃)₃SiO_{1/2} units to SiO₂ units is about 0.75 to 1. More specifically, the trimethylsiloxysilicate thus formed contains from about 2.4 to 2.9 weight percent hydroxyl groups which is formed by the reaction of the sodium salt of silicic acid, chlorotrimethylsilane, and isopropyl alcohol. The manufacture of trimethylsiloxysilicate is set forth in U.S. Pat. Nos. 2,676,182; 3,541,205; and 3,836,437, all of which are hereby incorporated by reference. Trimethylsiloxysilicate as described is available from GE Silicones under the tradename SR-1000, which is a solid particulate material. Also suitable is Dow Corning 749 which is a mixture of volatile cyclic silicone and trimethylsiloxysilicate.

The siloxane polymeric resins that may be used as porous particulates in the packaged composition of the invention may be made according to processes well known in the art. In general siloxane polymers are obtained by hydrolysis of silane monomers, preferably chlorosilanes. The chlorosilanes are hydrolyzed to silanols and then condensed to form siloxanes. For example, Q units are often made by hydrolyzing tetrachlorosilanes in aqueous or aqueous/alcoholic media to form the following: 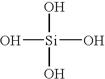
The above hydroxy substituted silane is then condensed or polymerized with other types of silanol substituted units such as: 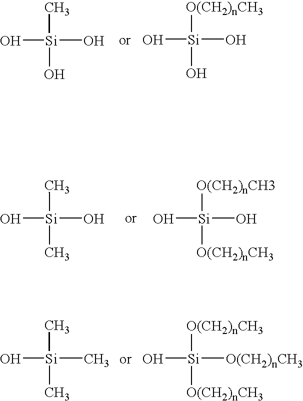
wherein n is 0-10, preferably 0-4.

Because the hydrolysis and condensation may take place in aqueous or aqueous/alcoholic media wherein the alcohols are preferably lower alkanols such as ethanol, propanol, or isopropanol, the units may have residual hydroxyl or alkoxy functionality as depicted above. Preferably, the resins are made by hydrolysis and condensation in aqueous/alcoholic media, which provides resins that have residual silanol and alkoxy functionality. In the case where the alcohol is ethanol, the result is a resin that has residual hydroxy or ethoxy functionality on the siloxane polymer. The silicone polymers that may be used in the packaged compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

4. Silica or Silicates

Also suitable for use as the porous particulates are various types of silicas or silicates. Examples of such silicates include those typically found in lamellar or porous form such as silica, filmed silica, calcium silicate, aluminum silicate, hydrated silica, magnesium aluminum silicate, magnesium trisilicate, silica silylate, or silicas that are substituted with hydrophobic or hydrophilic groups such as C₁₋₆ alkyl groups, C₁₋₆ alkoxy groups, and the like. One preferred type of porous particulate that may be used in the packaged compositions of the invention comprises silica.

5. Silicone Elastomers

Silicone elastomers may also be suitable porous particulates. Silicone elastomers are generally cross-linked organosiloxane compounds prepared by reacting a dimethyl methylhydrogen siloxane with a crosslinking group comprised of a siloxane having an alkylene group having terminal olefinic unsaturation or with an organic group having an alpha or omega diene. Examples of suitable silicone elastomers for use as thixotropic agents include Dow Corning 9040, sold by Dow Corning, and various elastomeric silicones sold by Shin-Etsu under the KSG tradename including KSG 15, KSG 16, KSG 19 and so on.

The packaged composition may contain one or more porous particulates. Preferred porous particulates include crosspolymers, more particularly those made from polymerized ethylenically unsaturated monomers, or cellulosics. Most preferred is methyl methacrylate crosspolymer either alone or in combination with sodium carboxylmethyl starch.

6. Lamellar Particulates

Also suitable are porous particulates in the lamellar form. The term “lamellar” means that the particulate exists in the form of interlayered sheets which have spaces between the sheets that permit fluid to be imbibed within. Examples of lamellar particulates include mica, talc, titanated mica, boron nitride, bentonite, diatomaceous earth, fuller's earth, hectorite, kaolin, montmorillonite, attapulgite, and the like.

B. The Solvent

Many ingredients are suitable for use as solvents in the packaged cosmetic composition, providing they are capable of being imbibed into the porous particulate and released from the particulate upon application of pressure as found with a suitable applicator.

Solvents may be aqueous or non-aqueous or a mixture of both. Only one solvent may be present, or more than one. The solvents must be present in an amount sufficient to cause the composition to form a liquid when applied to the keratinous surface, but not present in such a large amount that they cause the composition to be a liquid when in the container or in the resting state. Generally, solvents may be present in amounts ranging from 1-99%, preferably about 7-95%, more preferably about 10-85% by weight of the total composition.

1. Aqueous Solvents

A suitable aqueous solvent is water, either alone or in combination with ingredients that are soluble in water. In the most preferred embodiment of the invention the composition contains water either alone or in combination with other solvents that may be soluble or dispersible in water.

2. Non-Aqueous Solvents

Suitable non-aqueous solvents include water soluble, water dispersible, or oil soluble or oil dispersible ingredients. Suitable solvents include volatile solvents such as volatile silicones, volatile paraffinic hydrocarbons, volatile organic compounds; or non-volatile solvents such as non-volatile silicones, non-volatile organic oils, water soluble ingredients such as mono-, di-, or polyhydric alcohols, and the like.

(a). Volatile Silicones

Examples of suitable volatile silicones cyclic silicones are of the general formula: 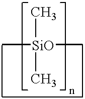
where n=3-6.

Linear volatile silicones are also suitable for use in the packaged cosmetic composition of the invention. Such silicones have the general formula:
(CH₃)₃Si—O—[Si(CH₃)₂—O]ₙ—Si(CH₃)₃
where n=0, 1, 2, 3, 4, 5, 6, or 7, preferably 0, 1, 2, 3, or 4. Such silicones include hexamethyldisiloxane, octamethyltrisiloxane, decamethyltetrasiloxane, dodecamethylpentasiloxane, and the like.

Such linear and cyclic volatile silicones are available from various commercial sources including Dow Corning Corporation and General Electric. The Dow Corning volatile silicones are sold under the tradenames Dow Coming 244, 245, 344, and 200 fluids, and have viscosities ranging from 0.5 to about 2.0 centistokes at 25° C. For example, hexamethyldisiloxane primarily comprises silicone having a viscosity of about 0.5 to 0.65 cst, while octamethyltrisiloxane primarily comprises a siloxane having a viscosity of about 1.0 cst, and decamethyltetrasiloxane comprises primarily a siloxane having a viscosity of 1.5 cst, all at 25° C.

(b). Paraffinic Hydrocarbons

Volatile paraffinic hydrocarbons that may be present in the packaged cosmetic compositions of the invention include various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 centipoise at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable. Examples of suitable volatile paraffinic hydrocarbons include isohexadecane, isododecane, or mixtures thereof.

( c). Non-Volatile Silicones

Examples of suitable non-volatile silicones that may be used in the packaged compositions of the invention include those disclosed in Cosmetics, Science and Technology 27-104 (Balsam and Sagarin ed. 1972); and U.S. Patent Nos. 4,202,879 and 5,069,897, both of which are hereby incorporated by references. Such silicones are generally liquids at room temperature. Further nonlimiting examples of such silicones include dimethicone, phenyl trimethicone, trimethylsiloxyphenyldimethicone, phenyl dimethicone, dimethicone copolyol, and so on.

(d). Esters

Also suitable are organic mono-, di-, or trimesters including but not limited to those set forth herein.

(i). Monoesters

Monoesters are defined as esters formed by the reaction of a monocarboxylic acid having the formula R—COOH, wherein R is a straight or branched chain saturated or unsaturated alkyl having 2 to 150 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, ie. may have from about 6 to 30 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

(ii). Diesters

Suitable diesters that may be used in the packaged compositions of the invention may be formed from the reaction of a dicarboxylic acid and an aliphatic or aromatic alcohol, or an aliphatic or aromatic alcohol having at least two hydroxyl groups with mono- or dicarboxylic acids. The carboxylic acids may contain from 2 to 150 carbon atoms, and may be in the straight or branched chain, saturated or unsaturated form. The carboxylic acids may be substituted with one or more hydroxyl groups. The aliphatic or aromatic alcohol may also contain 2 to 30 carbon atoms, and may be in the straight or branched chain, saturated, or unsaturated form. The aliphatic or aromatic alcohol may be substituted with one or more substituents such as hydroxyl. Preferably, one or more of the acid or alcohol is a fatty acid or alcohol, i.e. contains 14-22 carbon atoms. The carboxylic acids may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

(iii). Triesters

Suitable triesters comprise the reaction product of a tricarboxylic acid and an aliphatic or aromatic alcohol, or the reaction product of an aliphatic or aromatic alcohol having three or more hydroxyl groups with various mono-, di-, or tricarboxylic acids. As with the mono- and diesters mentioned above, the acid and alcohol contain 2 to 150 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, trioctydodecyl citrate dilinoleate, tridecyl behenate, tridecyl cocoate, tridecyl isononanoate, and so on.

(e). Hydrocarbon Oils.

Also suitable for use as solvents in the packaged cosmetic compositions of the invention may be one or more hydrocarbon oils that are liquid at room temperature. Suitable nonvolatile hydrocarbon oils include paraffins and olefins, preferably those having greater than 20 carbon atoms. Examples of such hydrocarbon oils include C₂₄₋₂₈ olefins, C₃₀₋₄₅ olefins, C₂₀₋₄₀ paraffins, hydrogenated polyisobutene, polyisobutene, mineral oil, pentahydrosqualene, squalene, squalane, and mixtures thereof.

(f). Lanolin Oil

Also suitable for use in the packaged cosmetic composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(g). Glyceryl Esters of Fatty Acids

The composition may comprise naturally occuring glyceryl esters of fatty acids, or triglycerides. Both vegetable and animal sources may be used. Examples of such oils include castor oil, lanolin oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(h). Fluorinated Oils

Also suitable as the oil are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589, 4,803,067, 5,183,588 all of which are hereby incorporated by reference, which are commercially available from Montefluos under the trademark Fomblin.

Fluoroguerbet esters are also suitable oils. The term “guerbet ester” means an ester which is formed by the reaction of a guerbet alcohol having the general formula: 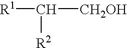
and a fluoroalcohol having the following general formula:
CF₃—(CF₂)ₙ—CH₂—CH₂—OH
wherein n is from 3 to 40.
with a carboxylic acid having the general formula:
R³COOH, or
HOOC—R³—COOH
wherein R¹, R², and R³ are each independently a straight or branched chain alkyl.

Another type of guerbet ester is a fluoro-guerbet ester which is formed by the reaction of a guerbet alcohol and carboxylic acid (as defined above), and a fluoroalcohol having the following general formula:
CF₃—(CF₂)ₙ—CH₂—CH₂—OH
wherein n is from 3 to 40.

Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. One type of such an ester is fluorooctyldodecyl meadowfoamate, sold under the tradename Silube GME-F by Siltech, Norcross Ga.

(i). Mono-, Di-, or Polyhydric Alcohols

Also suitable as solvents for use in the packaged compositions of the invention are one or more mono-, di-, or polyhydric alcohols, including but not limited to ethanol, isopropanol, propylene glycol, butylene glycol, ethylene glycol, glycerin, glycerol, and the like.

A sufficient portion of the solvents present in the packaged composition are imbibed within the interstices or channels of the porous particulate so as to cause the packaged composition to visually appear as a powder. However, it is not necessary that the solvent present be entirely found within the interstices or channels of the porous particulate. A portion of the solvents present may also coat the external surfaces of the particulates, be present as a binder for the particulates, or otherwise provide an emollient or tactile feel provided that the composition visually appears in the powder form.

C. Other Ingredients

The packaged composition may also comprise a variety of additional ingredients that provide coloration, improve the tactile properties, or provide special treatment effects or other beneficial effects.

1. Pigments and Powders

Preferred packaged cosmetic compositions of the invention contain pigments, powders, or mixtures thereof, which may or may not be porous in character. Suggested ranges of such ingredients are about 0.05-70%, preferably about 0.1-25%, more preferably about 0.5-20% by weight of the total composition. The pigments or powders may be dispersed or solubilized in the solvents used in the packaged composition, or they may be dispersed in the particulate form with the porous particulates.

Suitable pigments may be organic or inorganic. Examples of organic pigments include red, green, blue, yellow, violet, orange, and mixtures thereof. Also suitable are Lakes of such pigments, which means that the organic pigments are reacted with a metal salt such as calcium, aluminum, barium, zirconium, and the like to form salts. Preferred are aluminum Lakes of the organic pigments, which is where the organic pigment is reacted with aluminum to form the aluminum salt. Formation of the metal salt of the organic pigment will generally also convert the pigment from a water soluble pigment into a water insoluble pigment, and such pigments tend to become lipophilic in nature, meaning that they will exhibit affinity for lipophilic or oily ingredients in the composition. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors that are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51. Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like. Most preferred are Aluminum Lakes of the various red pigments mentioned herein.

Suitable yellows include wherein the yellow pigment is a pyrazole, monoazo, fluoran, xanthene, quinoline, or salt thereof. Suitable yellows include Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.

Also suitable are inorganic pigments that include iron oxides such as red, blue, black, green, and yellow; titanium dioxide, bismuth oxychloride, and the like. Preferred are iron oxides. While titanium dioxide is often classified as a pigment it is generally not used to provide color to a composition, but rather to mute the color provided by other organic or inorganic pigments.

It may also be desirable to include one or more powders in the claimed composition. If so, suggested ranges are about 0.001-40%, preferably about 0.05-35%, more preferably about 0.1-30% by weight of the total composition. Generally the term “powder” means non-pigmentitious particles (excluding titanium dioxide) that generally have a particle size ranging from about 0.02 to 200, preferably 0.5 to 100, microns. Such powders include, but are not limited to, micronized teflon, acrylate copolymers, silk powder, zinc laurate, zinc myristate, zinc rosinate, alumina, calcium carbonate, nylon, sericite, soy flour, tin oxide, titanium hydroxide, trimagnesium phosphate, walnut shell powder, or mixtures thereof. The particulates may also be in the fiber form, such as cellulose fibers, rayon fibers, nylon or silk fibers and the like. Such fibers are generally circular in cross-section and have a discernable length. Preferably the length ranges from 1 to 5 mm.

The above mentioned pigments, powders or fibers may be inherently lipophilic or hydrophilic. The term “lipophilic” when used in this context means that the particulates will exhibit an affinity for lipophilic ingredients, or will be soluble or dispersible in lipophilic ingredients such as nonpolar oils. The term “hydrophilic” when used in this context means that the particulates exhibit an affinity for, or are soluble or dispersible in, water. For example, many types of organic pigments are hydrophilic and will be soluble or dispersible in water. On the other hand, in some cases, if the organic pigments are reacted with metal salts to form Lakes, in some cases the pigments will then exhibit a more lipophilic character. In the packaged compositions of the invention, the pigments may be inherently lipophilic or inherently hydrophilic, depending on the properties desired. Further, if the pigments, particles or fibers are hydrophilic or if it is desired to increase their already lipophilic character, it may be desired to further surface treat the particulates with lecithin, amino acids, mineral oil, silicone oil or various other agents either alone or in combination, which coat the particulate surface and render the particles more lipophilic in nature. The term “lipophilic” means that the pigment or particles will be compatible with the lipophilic or oily phase of the composition. In the case of an emulsion, a lipophilic particle will have an affinity for the oily phase of the emulsion.

Particularly preferred for use in the packaged cosmetic compositions of the invention are iron oxide pigments and/or powders. The pigments or powders may be dispersed with the porous particulates in the powder composition or they may be solvated or dispersed in the solvent that is imbibed within the channels or interstices of the porous particulates.

2. Structuring Agents

It may be desirable to include one or more structuring agents in the packaged cosmetic compositions. Generally such structuring agents are solids or semi-solids at room temperature (25° C.) and may be waxes or similar types of polymeric thickeners. If present, suggested ranges are from about 0.01-45%, preferably about 0.1-40%, more preferably about 0.5-25% by weight of the total composition. Preferably such structuring agents, if present, will coat the porous particulates and/or pigments or powders and contribute to an improved tactile feel or emollience.

Suitable structuring agents include natural or synthetic waxes. A variety of waxes are suitable including animal, vegetable, mineral, or silicone waxes. Generally such waxes have a melting point ranging from about 28 to 125° C., preferably about 30 to 100° C. Examples of waxes include acacia, beeswax, ceresin, cetyl esters, fatty acids, fatty alcohols, flower wax, citrus wax, carnauba wax, jojoba wax, japan wax, polyethylene, microcrystalline wax, synthetic wax, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax; or fatty acids or esters such as hydroxystearic acids (for example 12-hydroxy stearic acid), tristearin, tribehenin, trihydroxystearin, and so on.

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone. Such silicones include, but are not limited to stearoxydimethicone, behenoxy dimethicone, stearyl dimethicone, cetearyl dimethicone, and so on. Suitable waxes are also set forth in U.S. Pat. No. 5,725,845, which is hereby incorporated by reference in its entirety.

Also suitable as structuring agents are various types of polyamides or silicone polyamides including those set forth in U.S. Patent Publication Nos. 2002/0114773 or 2003/0072730, both of which are hereby incorporated by reference in their entirety.

3. Botanicals

If desired, a wide variety of botanicals may be incorporated into the packaged cosmetic composition of the invention. Such botanicals may be in the form of extracts, proteins, or the like. If present, such botanicals may range from about 0.001-10%, preferably about 0.005 to 8%, more preferably about 0.01 to 5% by weight of the total composition. Suitable botanicals include, but are not limited to, plant extracts from flowers, vines, seeds, or leaves such as green tea, chamomile, rosemary, rose hips, orange, grapefruit, soy, yam, grape, grape seed, blueberry, apple, pineapple, date, and the like.

4. Preservatives

Preservatives may also be present in the packaged composition. If present, ranges from about 0.0001-5%, preferably about 0.0005-3%, more preferably about 0.001-2% by weight of the total composition is suggested. Suitable preservatives include parabens such as methyl, ethyl, or propyl parabens or mixtures thereof, urea, diazolidinyl urea, phenoxyethanol, and the like.

The packaged composition may also include a variety of other ingredients that may improve the beneficial properties of the composition, including but not limited to those set forth herein.

5. Film Forming Polymers

If desired, one or more film forming polymers may be solvated or dispersed in the solvent imbibed within the channels of the porous particulate or may be present as a coating for the porous particulates or pigments and powders that may be present. Suitable film forming polymers include those that are capable of forming a film on keratinous surfaces such as polyesters, polyurethanes, acrylate or methacrylate polymers. Such polymers may be incorporated into the solvent in dispersible or solvated form such that when the packaged composition is applied to the skin and the solvent expressed from the particulate, the film forming polymer present deposits on the skin and when dry improves skin properties such as tone and laxity, or improves transfer resistant or long wearing properties.

II. The Package

A. The Container

The packaged cosmetic composition of the invention comprises a container. A wide variety of containers are suitable so long as they are capable of storing and enabling dispensing of the composition in the powder form without applying sufficient pressure to the composition while it is in the resting state to cause the solvents within the porous particulates to exude from the particulates to form a liquid while the powder is contained in the container.

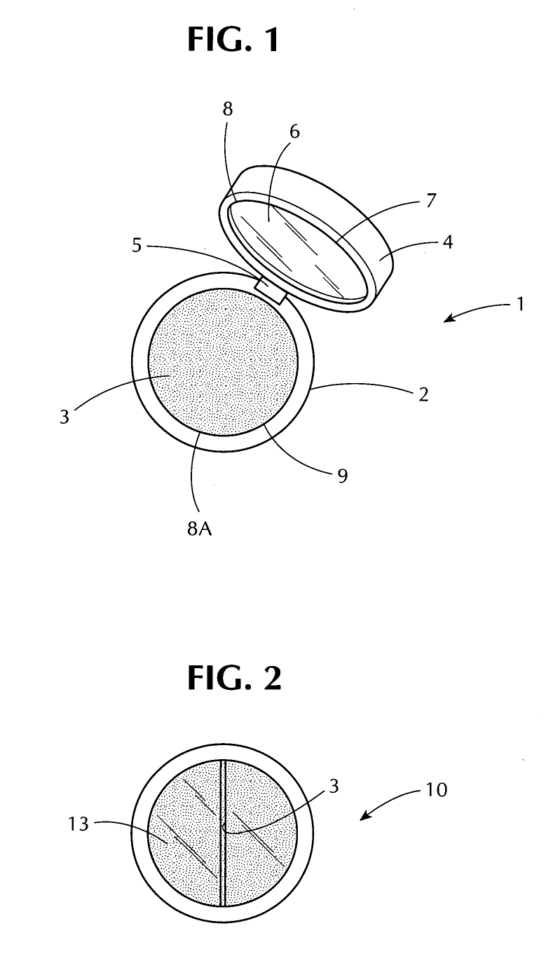

Suitable containers include those suitable for pressed or loose powders such as jars, wide neck bottles, compacts, boxes, and the like. The suitable container must facilitate pick up of the powder by the applicator. Suitable containers include, but are not limited to, those set forth in FIGS. 1 through

FIG. 1 depicts a compact case 1 that is suitable for containing the cosmetic composition used in the invention. The compact 1 has a base 2 for containing the cosmetic 3 and a cover 4 that is attached by a hinge 5 to the base 2. If desired, the cover 4 may have a mirror 6 on its inner surface, permitting the consumer to look at herself as she applies the powder with the various suitable applicators that will be further described herein. If desired, the compact may be hermetic, either by having a gasket or similar sealing means 7 on the cover 4 inner surface 8, or a similar gasket, foil liner, or sealing means 8A on the base inner surface 9. The compact 1 is closed when cover 4 is rotated on hinge 5 to close cover 4 on base 2 (compact in closed position not shown).

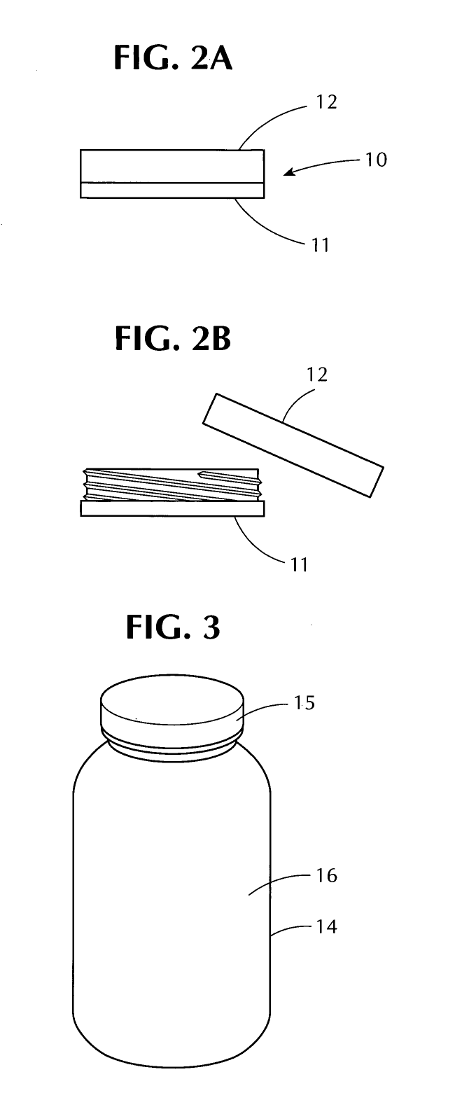

FIGS. 2 and 2A illustrate another type of cosmetic container suitable for containing the cosmetic 3, which is a compact 10 having a base portion 11 for containing the cosmetic composition 3 and a lid 12 that is affixed to the base portion 11 by engagements that mate with similar engagements on the base portion 11. Most preferably, the lid 12 is a screw cap that is affixed to base portion 11 by screwing lid 12 on base portion 11. Although the compact 10 of FIG. 2 is illustrated as having a window 13 in the lid 12 that permits viewing of the cosmetic composition 3 when it is in the compact 10, this is not a necessary feature and the lid 12 could be entirely made of an opaque thermoplastic or other material that does not permit viewing of the cosmetic composition 3 in the compact 10.

FIG. 3 illustrates another type of container suitable for containing the cosmetic composition 3 used in the invention. This container is ajar 14 having a lid 15 and a container 16 portion. The lid 15 is removable from the container portion 16 by disengaging, such as by screwing on and off in the typical manner.

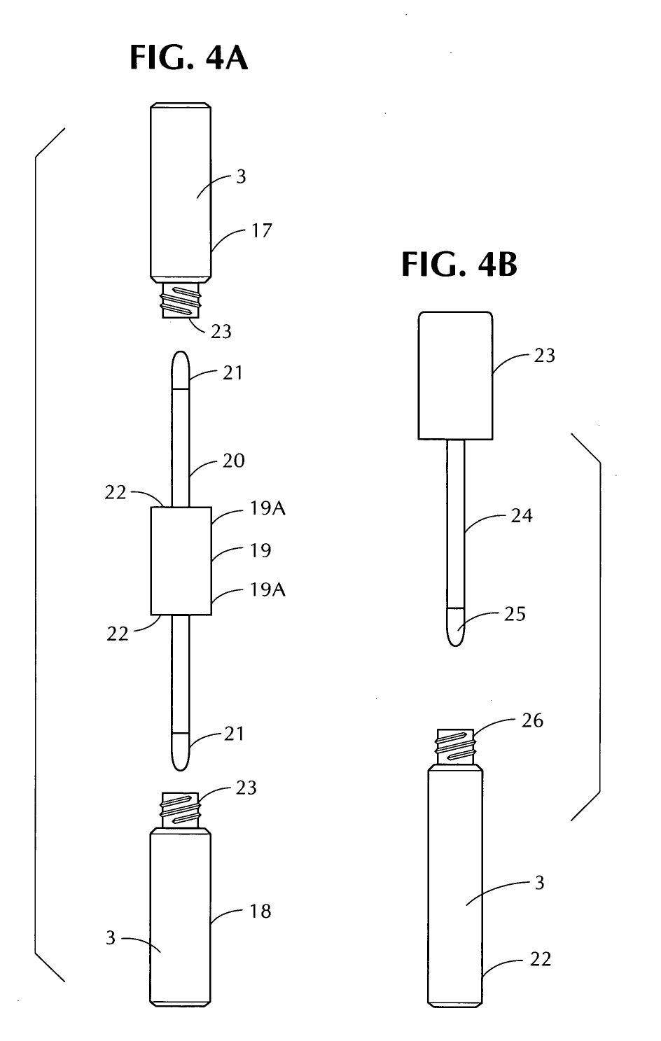

FIG. 4 depicts various types of cylindrical containers suitable for containing the cosmetic composition used in the invention.

FIG. 4A illustrates a dual ended container where the container on one end 17 is a cylindrical container which contains one type of cosmetic composition and the cylindrical container on the other end 18 contains another type of cosmetic composition where, in accordance with the invention, at least one of the cosmetic compositions in at least one of the containers 17 or 18 is the powder-to-liquid-to-powder composition of the invention, while the other container may contain a second powder-to-liquid-to-powder composition or another type of composition that is usable with the powder-to-liquid-to-powder composition. Examples of such combinations include a powder-to-liquid-to-powder lipstick in container 17 and in container 18 a lip gloss suitable for use with the lipstick in container 17. Or the dual ended container of FIG. 4A may contain two different powder-to-liquid-to-powder eyeshadows in different colors, separately stored in containers 17 and 18. The dual ended container of FIG. 4A may also contain foundation and concealer or two different concealers in each of containers 17 and 18, both in the powder-to-liquid-to-powder form of the invention. Another suitable embodiment may be a combination of blusher and concealer, concealer and eyeshadow, or a blemish touch up and concealer, or any number of combinations in containers 17 and 18. A suitable closure for the dual ended container of FIG. 4A is dual closure 19 having a rod 20 and applicator 21 on either end. Both ends 22 of the dual closure 19 form a cap 19A that is engagable with neck 23 found on containers 17 and 18 so each container 17 can be opened by disengaging cap 19A from container 17 or 18 when desired. When container 17 or 18 is disengaged from cap 19A the applicator 21, which rests in the cosmetic 3 found in container 17 or 18 can be removed from the container 17 or 18, and the applicator 21, which rests in the cosmetic 3 will pick it up so that the cosmetic 3 can be applied to the desired keratinous surface, which may be skin, nails, or hair, but is preferably a skin surface such as lips, cheeks, eyelids, or any area of the face. The term “pick up” or “pick it up” when used herein means that the applicator is capable of picking up the cosmetic and retaining it and releasing it when the applicator is used to apply the cosmetic to the keratinous surface.

FIG. 4B illustrates another type of container, which is cylindrical container 22 having a cap 23 to which is attached a rod 24 and applicator 25. The cap 23 is engageable with the container 22 by engaging with neck 26 by similar engaging threads on the inner surface of cap 23 (not shown). Cosmetic 3 is contained within container 22, and may be blush, eyeshadow, concealer, blemish treatment, powder, foundation, and the like.

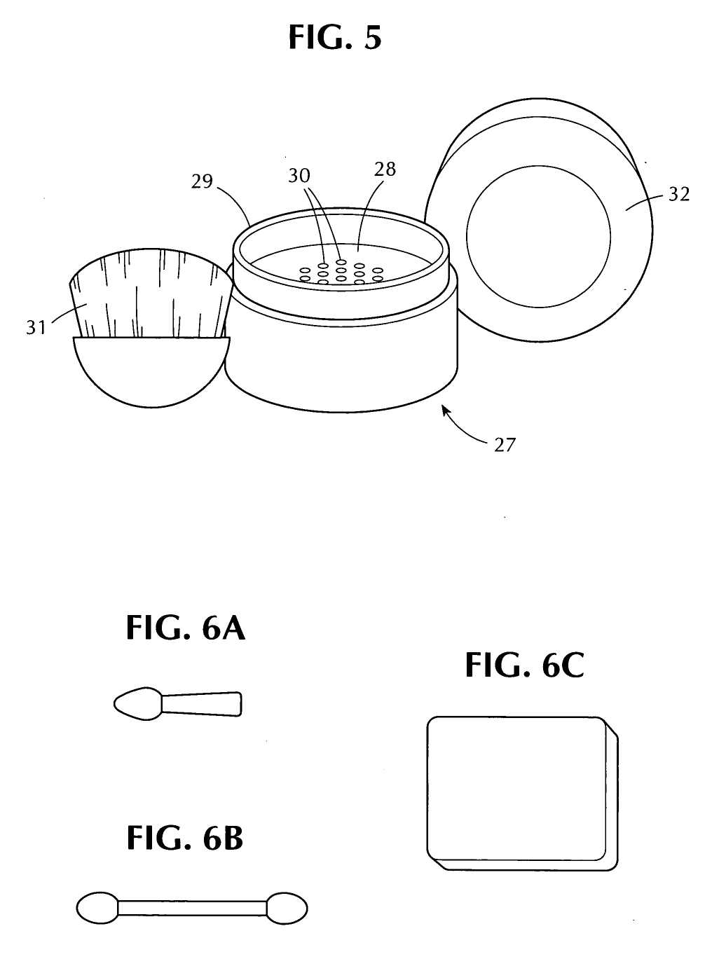

FIG. 5 illustrates one preferred type of container 27 for containing the packaged cosmetic composition of the invention. The container 27 is ajar in which the powder cosmetic composition is stored. A porous lid 28 is affixed to the container 27 side wall 29 and rests over the powder cosmetic composition (not shown) that is found within the container 27. The porous lid 28 contains one or more holes 30 such that the container 27 can be shaken slightly from the side to cause the powder to be dispensed from the holes 30 onto the surface of the porous lid 28, where the powder is then available for pick up and application by the applicator 31. The container 27 has a cover 32 that is affixed to the container 27 when the package is not in use. When the user desires to apply the powder composition, the cover 32 is removed, the container 27 is shaken to cause the powder to be dispensed from the container 27 through the holes 30 in the porous lid 28 so that the applicator 31 can be used to pick up the powder and spread it on the desired keratinous surface.

There are various other types of containers that may be used for the package in the packaged cosmetic composition and methods of the invention. The containers may be made of glass, various thermoplastic materials such as polyethylene, polypropylene, nylon, PET (polyethylene terephthalate), polystyrene, or copolymers of ethylene, propylene, terephthalate, styrene, acrylates, methacrylates, and the like. The containers may be in the form of cosmetic compacts, vials, jars, bottles, and the like. Preferably, the package used for the packaged cosmetic composition of the invention is hermetically sealed to keep the water or other solvents that may be used from evaporating from the powder composition. The hermetic seal may be achieved by a simple screw cap having a gasket or similar seal, or in the case of a compact, a gasket around the top edge, bottom edge, or both edges such that the gasket fits tightly against the surface it is compressed against to form an air tight seal.

B. The Applicator

A variety of suitable applicators may be used provided they have certain pick up and application properties. With respect to pick up, the applicator must be capable of picking up the right amount of the powder composition without causing the solvent present in the porous particulate to be expressed from the particulate on pick up so that the powder composition does not turn into a liquid prior to application to the desired keratinous surface. On the other hand, the applicator must be capable of exerting enough pressure on the porous particulates in the powder composition as they are being applied to the keratinous surface to thereby cause the particulates to release sufficient solvent to form a liquid when applied to the keratinous surface.

Suitable applicators may include foam or cloth pads or sponges, foam tipped or flocked applicators which may be affixed to a rod, brushes, plastic tips, and the like. Suitable foams include, but are not limited to polyurethane or similar materials that may be in the open cell or closed cell foam configuration. The term “open cell” means that at least a portion of the cells in the foam have interconnected channels. The term “closed cell” means that at least a portion of the cells in the foam are separate and distinct, and do not form interconnected channels. Also suitable for use in this type of applicator are various types of natural or synthetic fibers such as cotton, acrylics, rayon, and the like.

If in the brush form the fibers may be made from thermoplastic materials or animal hair. Suitable thermoplastic materials include, but are not limited to nylon, polyester, polyethylene, polypropylene, PET, and so on. Suitable animal hair fibers are goat, pony, dog, llama, sable, mink, and the like.

Digital application may be suitable for applying the packaged cosmetic composition, provided that not too much of he powder is picked up with the fingers. Because of the wide variability in the amount of powder picked up with the fingers from consumer to consumer, the fingers may not be the most desired applicator. In addition, digital application does not always provide uniform application and may leave product residue on the hands.

FIG. 6 depicts a number of foam, cloth, or sponge type applicators that may be used to apply the powdered composition of the invention.

FIG. 6A depicts a rod having a foam tip applicator on the end, such as the type that may be found in a cosmetic compact. Such applicators are often used to apply cosmetics such as eyeshadow, or lip color.

FIG. 6B depicts a dual ended rod having a foam tip applicator on both ends. This type of applicator is also frequently used in cosmetic compacts, and can be used to apply eyeshadow, blush, or other types of color cosmetics.

FIG. 6C depicts a sponge type applicator that may be made from foam, fabric, or a similar material. These types of applicators are often found in cosmetic compacts like those depicted in FIG. 1.

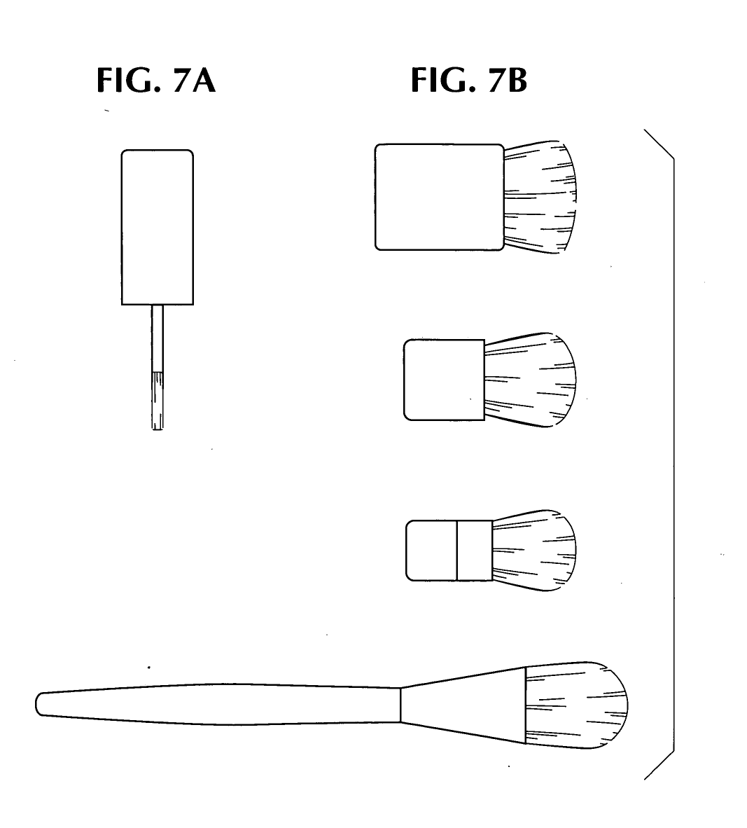

FIG. 7 illustrates various types of brushes that may be used to apply the packaged cosmetic composition of the invention.

FIG. 7A depicts a brush with cap that is suitable for use in applying the packaged cosmetic composition of the invention to small surfaces such as cuticles.

FIG. 7B depicts various types of brushes that can be used to apply the packaged cosmetic composition. As noted above, the brushes may be used with any one or more of the cosmetic containers depicted in FIGS. 1-5 to apply the composition to the desired keratinous surface, which is most preferably skin.

The term “keratinous surface” when used herein means skin, hair, or nails. The term “skin” when used in accordance with this invention includes lips, eyelids, cuticles, and similar skin surfaces.

III. The Methods

A. Improving Skin Laxity or Tone

The invention comprises a method for improving laxity and/or tone of skin comprising applying to said skin a composition comprising at least one porous particulate having contained within at least one liquid solvent expressible from the particulate upon application of pressure, and upon application, providing pressure sufficient to express the liquid solvent from the porous particulate to form a liquid film on the skin that, when dry, improves the laxity and/or tone of the skin.

When the solvent is expressed from the porous particulate upon application to the skin, it forms a film that, when dry, tightens on the skin to provide improvements in skin tone and laxity. If desired, the effect on skin tone and laxity can be even further improved by including ingredients that are soluble or dispersible in the solvent or on the porous particulates that provide skin firming or toning effects. Examples of such ingredients include, but are not limited to those described above with respect to the composition, including but not limited to botanicals, film forming polymers, and the like.

B. Improving Transfer Resistance

The invention is also directed to a method for providing a transfer resistant or long wearing finish on skin by applying a color cosmetic composition containing at least one porous particulate having contained therein at least one liquid solvent expressible from the particulate upon application of pressure, and upon application, providing pressure sufficient to express the liquid solvent from the porous particulate to form a liquid film on the skin that dries to a transfer resistant or long wearing finish.

In the above method, the porous particulates, the solvent, or any of the other ingredients may have properties that contribute to providing improved transfer resistance. In the most preferred embodiment, porous particulates such as silicone resins and/or starch may be used. Such silicone resins and starch may improve the transfer resistant or long wearing properties of the composition particularly when combined with one or more volatile solvents. Typically, in such a case, the solvents, some of which are volatile, are expressed from the porous particulate upon application of the powder composition to the skin. The composition forms a liquid upon application, which then dries to a transfer resistant or long wearing film. Preferably the solvent component includes volatile linear or cylic silicones or volatile paraffinic hydrocarbons. While silicone resins are excellent porous particulates for providing transfer resistance, other types of porous particulates or film forming polymers may be suitable. The transfer resistant or long wearing property may be attributable, at least in part, to one or more of the porous particulates, or to one or more of the other ingredients which are present in the composition.

The invention will be further described in connection with the following Examples, which are set forth for the purposes of illustration only.

### EXAMPLE 1

A cosmetic composition to be used with the package and the method of the invention was made as follows:

| | w/w % |
|---|---|
| Sodium carboxymethyl starch | 5.00 |
| Mica & dimethicone & trimethylsiloxysilicate | 3.60 |
| Talc & dimethicone | 4.60 |
| Talc and perfluoropolymethylisopropyl ether | 9.80 |
| Methylmethacrylate crosspolymer | 3.00 |
| Titanium dioxide, trimethoxycaprylyl silane, PMMA | 4.20 |
| Methyl paraben | 0.20 |
| Propyl paraben | 0.10 |
| Black iron oxide | 0.10 |
| Red iron oxide | 0.26 |
| Yellow iron oxide | 0.72 |
| Water | 65.32 |
| Diazolidinyl urea | 0.20 |

The composition was prepared by blending the particulate ingredients in an Osterizer blender to provide a uniform powder and develop the color of the powder. The blended mixture is then mixed in a vessel using a t-shaped blade and a laboratory stirring mixer. The aqueous phase is then added slowly with high speed mixing with tip speeds of between 200 to 400 feet/minute. The product is mixed until the composition visually appears as a powder.

We claim:

1. A packaged powder-to-liquid-to-powder cosmetic composition wherein the composition in the package is in the powder form and comprises a powder-to-liquid effective amount of at least one porous particulate having contained within at least one liquid solvent expressible from the particulate upon application of pressure; and the package comprises a container, and an applicator for the composition that is operable to pick up the composition in its powder form and upon application of the powder to the keratinous surface, exert pressure sufficient to express the liquid from the porous particulate causing the powder to form a liquid upon application to the keratinous surface, which dries to a powder finish.

2. The packaged composition of claim 1 wherein the porous particulate is polymeric.

3. The packaged composition of claim 1 wherein the porous particulate comprises cellulosics, crosspolymers, resins, silica, silicates, silicone elastomers, or mixtures thereof.

4. The packaged cosmetic composition of claim 3 wherein the porous particulate comprises cellulosics, crosspolymers, or mixtures thereof.

5. The packaged cosmetic composition of claim 4 wherein the cellulosics are homo- or copolymers of cellulose monomers that may be substituted with alkali or alkaline earth metals or C₁₋₁₀ alkyl or alkoxy groups.

6. The composition of claim 6 wherein the cellulosic comprises starch or starch substituted with C₁₋₁₀ alkyl or alkoxy groups or alkali or alkaline earth metals.

7. The composition of claim 1 wherein the porous particulate is starch, hydroxypropyl starch, hydroxyethyl starch, sodium carboxymethyl starch, aluminum starch octenylsuccinate, corn starch, rice starch, microcrystalline cellulose, maltodextrin, aluminum starch, dextran, glyceryl starch, or mixtures thereof.

8. The packaged cosmetic composition of claim 1 wherein the porous particulate comprises a crosspolymer.

9. The packaged cosmetic composition of claim 8 wherein the crosspolymer is a polymer of ethylenically unsaturated monomers.

10. The packaged cosmetic composition of claim 9 wherein the crosspolymer is a polymer of acrylate or methacrylate monomers.

11. The packaged cosmetic composition of claim 10 wherein the crosspolymer comprises methyl methacrylate crosspolymer.

12. The packaged cosmetic composition of claim 1 wherein the porous particulates comprise a mixture of cellulosics and crosspolymers from ethylenically unsaturated monomers.

13. The packaged cosmetic composition of claim 1 comprising about 2-98% by weight of the total composition of porous particulates.

14. The packaged cosmetic composition of claim 1 wherein the porous particulates have a particle size ranging from about 20 to 1000 microns.

15. The packaged cosmetic composition of claim 1 wherein the porous particulates have a porosity ranging from about 50 to 500 ml/100 grams.

16. The packaged cosmetic composition of claim 1 wherein the porous particulates have a density ranging from about 0.08 to 0.55 g/cm³.

17. The packaged cosmetic composition of claim 1 wherein the container comprises a compact, jar or bottle.

18. The packaged cosmetic composition of claim 1 wherein the applicator comprises a brush, foam applicator, or sponge.

19. The packaged cosmetic composition of claim 1 wherein the solvent comprises water either alone or in combination with one or more non-aqueous solvents which are silicones, hydrocarbons, esters, mono-, di-, or polyhydric alcohols, or mixtures thereof.

20. The packaged cosmetic composition of claim 1 wherein the applicator is a brush.

21. The packaged cosmetic composition of claim 1 which is a foundation, concealer, lipstick, eyeshadow, blemish treatment, or blusher.

22. A packaged powder-to-liquid-to-powder cosmetic composition wherein the composition in the package is in the powder form and comprises a powder-to-liquid effective amount of a mixture of porous particulates, at least one of which has transfer resistant or long wearing properties, wherein at least one of the porous particulates has contained within at least one liquid volatile solvent expressible from the particulate upon application of pressure; and the package comprises a container, and a brush applicator for the composition that is operable to pick up the composition in its powder form and upon application of the powder to the keratinous surface, exert pressure sufficient to express the liquid from the porous particulate causing the powder to form a liquid upon application to the keratinous surface, which dries to a transfer resistant powder finish.

23. The packaged cosmetic composition of claim 22 wherein at least one of the porous particulates comprises a silicone resin.

24. The packaged cosmetic composition of claim 22 wherein the container is a jar having a lid with one or more pores.

25. The packaged cosmetic composition of claim 24 wherein the powder can be shaken from the container through the lid with one or more pores upon manipulation of the container.

26. The packaged cosmetic composition of claim 25 wherein the applicator is a brush made from synthetic fibers.

27. A method for improving laxity and/or tone of skin comprising applying to said skin a composition one porous particulate having contained within at least one liquid solvent expressible from the particulate upon application of pressure, and upon application, providing pressure sufficient to express the liquid solvent from the porous particulate to form a liquid film on the skin that, when dry, improves the laxity and/or tone of the skin.

28. A method for providing a transfer resistant or long wearing finish on skin by applying a color cosmetic composition containing at least one porous particulate having contained therein at least one liquid solvent expressible from the particulate upon application of pressure, and upon application, providing pressure sufficient to express the liquid solvent from the porous particulate to form a liquid film on the skin that dries to a transfer resistant or long wearing finish.
